(19) United States
(12) Patent Application Publication
(10) Pub. No.: US 2025/0235401 A1
(43) Pub. Date: Jul. 24, 2025

(54) OIL-IN-WATER NANOEMULSIONS AND METHODS OF PRODUCTION AND USE THEREOF

(71) Applicant: Insucaps Limited, Dublin (IE)

(72) Inventor: Sinead Bleiel, Dublin (IE)

(21) Appl. No.: 18/833,380

(22) PCT Filed: Jan. 27, 2023
(86) PCT No.: PCT/EP2023/052093

§ 371 Date: Jul. 25, 2024

(30) Foreign Application Priority Data
Jan. 27, 2022 (GB) 2201084.7

(51) Int. Cl. A61K 9/107; A61K 9/50; A61K 31/7105; A61K 38/22; A61K 39/00

(52) U.S. Cl. CPC A61K 9/1075; A61K 9/5052; A61K 9/5089; A61K 31/7105; A61K 38/22; A61K 39/00

(57) ABSTRACT

An oil in water nanoemulsion has an oil phase and an aqueous phase and comprises active pharmaceutical ingredient (API), edible oil, denatured plant protein, surfactant, and water, in which the API is contained in the oil phase. The API comprises an RNA molecule or a hydrophobic drug. The oil is high oleic oil. The oil in water nanoemulsion may be combined with a suspension of denatured plant protein and a calcium salt chelating agent to form microparticles or microcapsules comprising the nanoemulsion or the oil droplets of the nanoemulsion encapsulated within a denatured plant protein matrix. The microcapsules can be ingested orally and pass through the stomach to the ileum where the protein matrix breaks down to release the oil droplets containing the API, which is then absorbed.

### FIELD OF THE INVENTION

The present invention relates to an oil in water nanoemulsion and uses thereof to make microparticles comprising active pharmaceutical ingredients such as hydrophobic drugs and immunogens encapsulated within a protein matrix.

### SUMMARY OF THE INVENTION

The disclosure generally relates to a nanoemulsion containing an active pharmaceutical ingredient (API) in the nano-sized oil droplets of the nanoemulsion, and microparticles containing the nanoemulsion that are capable of passing through the stomach intact and releasing their contents in the ileum, where the oil droplets containing the API are released and absorbed by the cell lining of the ileum. The API may be a hydrophobic drug or an immunogen for use in vaccine therapy. The methods and products of the invention enable these API's to be administered orally, obviating the need for the drugs to be injected.

In a first aspect, the invention provides an oil in water (o/w) nanoemulsion having an oil phase and an aqueous phase and comprising:
active pharmaceutical ingredient (API);
edible oil;
denatured plant protein;
surfactant; and
water,
in which the API is contained in the oil phase.

In any embodiment, the API comprises an immunogen suitable for use in a vaccine therapy. The immunogen may be a protein, polypeptide, protein subunit, peptide, hormone, or cell extract.

In any embodiment, the API comprises a nucleic acid encoding an immunogen suitable for use in a vaccine therapy. Typically, the nucleic acid is a messenger RNA molecule (mRNA) and ideally a long mRNA molecule.

In any embodiment, the API comprises a hydrophobic drug.

In any embodiment, the API comprises an antigen suitable for use in a vaccine therapy.

In any embodiment, the edible oil is high oleic oil. High oleic oil is any oil that is high in monounsaturated fats. Examples include corn, canola and sunflower oil.

In any embodiment, the oil in water nanoemulsion comprises 30 to 70% by weight water or aqueous buffer and 30 to 70% by weight solid ingredients.

In any embodiment, the oil in water nanoemulsion comprises 40 to 60% by weight water or aqueous buffer and 40 to 60% by weight solid ingredients.

In any embodiment, the solid ingredients comprise by weight:
55 to 95% API;
2 to 20% edible oil;
2 to 20% denatured plant protein;
1 to 5% surfactant.

In any embodiment, the solid ingredients comprise by weight:
70 to 90% API;
2 to 10% edible oil;
5 to 15% denatured plant protein;
1 to 5% surfactant.

In any embodiment, the solid ingredients comprise by weight 0 to 10% or 3 to 7% Immunoglobulin A.

In any embodiment, the oil droplets in the oil in water nanoemulsion have an average dimension of 10 to 100 nm or 25 to 60 nm.

In any embodiment, the surfactant comprises a non-ionic surfactant, for example a polysorbate-type non-ionic surfactant. Examples include Tween, Polysorbate 20 and Polysorbate 60.

In any embodiment, the plant protein is pea protein.

In any embodiment the plant protein is mung bean protein.

In any embodiment, the denatured plant protein is denatured, UHT-treated, plant protein.

In any embodiment, the oil in water (o/w) nanoemulsion comprises an adjuvant for a vaccine.

In another aspect, the invention provides a composition comprising:
5 to 50% by weight of an oil in water nanoemulsion according to the invention; and
50 to 95% by weight of a denatured plant protein suspension.

In another aspect, the invention provides a composition comprising:
5 to 20% by weight of an oil in water nanoemulsion according to the invention; and
80 to 95% by weight of a denatured plant protein suspension.

In any embodiment, the composition comprises a simple carbohydrate.

In any embodiment, the composition comprises a calcium salt chelating agent.

In another aspect, the invention provides a method of forming a microparticle comprising an API encapsulated in a denatured plant protein matrix, comprising the steps of
preparing a protein suspension comprising denatured plant protein;
combining the protein suspension and the API to form a mixture;
treating the mixture to form a microparticle comprising API encapsulated in a denatured plant protein matrix, in which the treating step comprises polymerising the denatured plant protein matrix with a calcium salt or forming the microparticle by spray englobing on a fluidised bed dryer; and
drying the microparticle.

In one embodiment, the API is provided in the form of an oil-in-water emulsion in which the API is contained in the oil phase of the oil-in-water emulsion.

In another embodiment, in which the API is not stable in oil, the API may be provided as a solution or suspension of API in buffered solution (e.g. a phosphate buffer).

In another embodiment, the API is distributed throughout a carrier powder (for example, native plant protein or starch)

In any embodiment, the protein suspension comprises a simple carbohydrate (e.g. 1-6% by weight).

In any embodiment, the protein suspension comprising denatured plant protein is prepared by preparing a suspension of plant protein, and heating the suspension of plant protein to achieve a heat treatment of F0=3. This results in denaturation of the plant protein (e.g. to at least 80% denaturation) and kills pathogenic/spoilage bacterial spores in the suspension. The heat treatment also lowers the viscosity of the protein suspension making it easier to process. The term “F0” is defined as the number of equivalent minutes of steam sterilization at 250° F. (121° C.) delivered to a load (product).

In any embodiment, the suspension is subjected to ultra-heat treatment (UHT).

In any embodiment, the suspension is heated to about 138.5° C.×about 3-4 secs.

In any embodiment, the combining step comprises extruding the protein suspension and the API (e.g. an oil in water nanoemulsion) to form microdroplets, in which the treating step comprises curing/polymerising the extruded microdroplets in a curing bath comprising a calcium salt to form microparticles (also referred to as microcapsules).

In any embodiment, the protein suspension and the API are mixed prior to extrusion through a single nozzle extruder.

In any embodiment, the extruding employs a concentric nozzle extruder in which the protein suspension is extruded through the outer nozzle and the API (e.g. an oil in water nanoemulsion) is simultaneously extruded through the inner nozzle.

In any embodiment, the curing bath comprises a calcium citrate buffer having a pH of 5 to 6.5.

In any embodiment, the curing bath comprises a calcium citrate buffer having a molarity of 0.05 to 0.15 M.

In an alternative embodiment to the extrusion/curing bath embodiment, the combining step comprising mixing the protein suspension and API (e.g. an oil in water nanoemulsion of the invention) to form a mixture, in which the treating step comprises adding a calcium salt chelating agent to the mixture to gel the mixture, drying the gelled mixture typically by freeze-drying or vacuum drying to form a solid, and typically size reducing the dried solid to form microparticles. This process is referred to herein as gel entrapment.

In any embodiment, the solid is size reduced using a high shear solid separator.

In any embodiment, the calcium salt chelating agent comprises a calcium citrate buffer having a pH of 5 to 6.5.

In any embodiment, the calcium salt chelating agent comprises a calcium citrate buffer having a molarity of 0.05 to 0.15 M.

In any embodiment, the calcium salt chelating agent is added to the mixture at a volumetric ratio of 1:100 to 1:300.

In any embodiment, the protein suspension comprises 10 to 15% denatured plant protein (by weight).

In any embodiment, the protein suspension comprises 0.5 to 5.0% simple carbohydrate.

In any embodiment, the combining step comprises combining the protein suspension and the oil in water nanoemulsion at a denatured plant protein to oil in water nanoemulsion weight ratio of 1:5 to 1:25.

In any embodiment, the simple carbohydrate comprises maltodextrin and glucose.

In any embodiment, the method comprises forming the microparticles by spray englobing in which the combining and treating steps are typically performed on a fluidised bed dryer.

In any embodiment, the method comprises adding a carrier material and an API to a bed of a fluidised bed dryer, fluidising the carrier material and API, spraying a first coating material onto the fluidised carrier material and API to produce a microparticles having a API and carrier contained within a shell of first coating material, and drying the microparticles.

In any embodiment, a simple sugar is sprayed into the fluidised bed dryer prior to the first coating material being sprayed into the fluidised bed dryer, wherein the simple sugar produces granules and the first coating material coats the granules.

In any embodiment, the first coating material is selected from denatured plant protein, oil, and a simple sugar such as maltodextrin.

In any embodiment, the method comprises spraying a second coating material on the shell of first coating material, wherein at least one of the first and second coating materials is denatured plant protein.

In any embodiment, the method comprises spraying a chelating salt on the denatured plant protein coating.

The following microparticles are envisaged:

### Microparticle 1

Core of API and carrier material
Coating of polymerised denatured plant protein

### Microparticle 2

Core of API and carrier material
Coating of non-polymerised dried denatured plant protein

### Microparticle 3

Core of API and carrier material
Inner coating of polymerised denatured plant protein
Outer coating of non-polymerised dried denatured plant protein
Intermediate coating of oil.

### Microparticle 4

Core of API and carrier material
Inner coating of polymerised denatured plant protein
Outer coating of polymerised denatured plant protein
First intermediate coating of oil.
Second intermediate coating of non-polymerised dried denatured plant protein

In one embodiment, the spray englobing comprises the steps of:
(a) adding a carrier material (e.g. a native protein), an API, and optionally a simple sugar, to a bed of a fluidised bed drying chamber;
(b) fluidising and heating the carrier material and API to form a first fluidised powder;
(c) spraying the denatured protein suspension onto the fluidised bed at elevated pressure (e.g. 2-4.5 Bar) to provide a second fluidised powder;
(d) drying the second fluidised powder on the fluidised bed to reduce the moisture content of the second fluidised powder (e.g. reduce moisture content by at least 30%, 40% or 50%);
(e) spraying an englobing component on to the second fluidised powder to form a third fluidised powder, in which the englobing component is selected from a chelating salt (e.g. a calcium salt or a magnesium salt); an edible oil, and a simple sugar;
(f) drying the third fluidised powder on the fluidised bed to further reduce the moisture content of the fluidised powder (e.g. further reduce moisture content by at least 10%, 20% or 25%);
(g) spraying the denatured protein suspension onto the fluidised bed at elevated pressure (e.g. 2-4.5 Bar) to provide a fourth fluidised powder comprising microparticles; and
optionally, further drying the fourth fluidised powder, typically to a moisture content of less than 10%, 8%, or 5%.

In any embodiment, the process comprises at least 2 or 3 rounds of steps (f) and (g).

In any embodiment, when the englobing component is an edible oil, the process includes an additional step of spraying a chelating salt onto the third fluidised powder prior to the second drying step. The method may comprise at least 2 or 3 rounds of spraying the edible oil and then spraying the chelating salt.

Generally, the spraying steps referenced above are top spraying, e.g. the component is sprayed from above the fluidised bed.

In any embodiment, the method comprises the following additional steps:
(h) bottom spraying denatured protein suspension onto the fourth fluidised mixture to form a fifth fluidised powder;
(i) drying the fifth fluidised powder on the fluidised bed to further reduce the moisture content of the fluidised powder (e.g. further reduce moisture content by at least 10%, 20% or 25%);
(j) bottom spraying an englobing component on to the fifth fluidised powder to form a sixth fluidised powder, in which the englobing component is selected from a chelating salt (e.g. a calcium salt or a magnesium salt) and an edible oil;
(k) drying the sixth fluidised powder on the fluidised bed to further reduce the moisture content of the fluidised powder (e.g. further reduce moisture content by at least 10%, 20% or 25%); and
(l) optionally, further drying the fourth fluidised powder, typically to a moisture content of less than 10%, 8%, or 5%.

In any embodiment, the process comprises at least 2 or 3 rounds of steps (j) and (k).

In any embodiment, when the englobing component of step (j) is an edible oil, the process includes an additional step of spraying a chelating salt onto the sixth fluidised powder prior to step (k). The method may comprise at least 2 or 3 rounds of spraying the edible oil and then spraying the chelating salt.

In any embodiment, the fluidised bed is fluidised with an airflow of 110 to 300 m³/hour.

In any embodiment, the fluidised bed chamber is heated during all or part of the method, typically to 35 to 45° C.

In any embodiment, the protein solution is sprayed into the fluidised bed at an elevated spray nozzle pressure, e.g. 2 to 4.5 Bar.

In any embodiment, the simple sugar is added to the second fluidised powder in an amount of 10-20% total dry solids.

In any embodiment, the simple sugar is or comprises maltodextrin.

In any embodiment, the chelating salt is added as a 0.2-0.4 M solution.

In any embodiment, the edible oil is added to the second fluidised powder in an amount of 5-15% total dry solids.

In any embodiment, step (a) comprises adding the carrier material and the API to the bed of a fluidised bed drying chamber in a weight ratio of 1:5 to 5:1.

The carrier material is generally native protein, but may also be a carbohydrate such as maltodextrin or starch, a prebiotic fibre powder or any colloid.

In any embodiment, the spray englobing comprises the steps of:
fluidising a carrier mater, an API and a chelating salt on a fluidised bed of a fluidised bed drying chamber;
spraying an edible oil into the fluidised bed drying chamber;
spraying a suspension of denatured protein into the fluidised bed drying chamber,
   whereby the chelating salt reacts with the denatured plant protein to polymerise the protein and form agglomerated microparticles having a polymerised denatured protein coat and core comprising API and carrier material.

In any embodiment, the carrier material is added first to the fluidised bed, the API and chelating salt is added to the fluidised bed after the carrier material.

In any embodiment, the carrier material is fluidised at elevated temperature (e.g. 30-40° C.) prior to the addition of the API and chelating salt.

In any embodiment, the carrier material, API, and chelating salt are fluidised at elevated temperature (e.g. 30-40° C.) prior to the addition of the edible oil to form a fluidised mixture.

In any embodiment, a simple sugar is sprayed onto the fluidised mixture prior to the edible oil to promote granulation of the mixture.

In any embodiment, the simple sugar is sprayed into the fluidised bed chamber at high flow rate (e.g. 23 to 30 RPM), low airflow (e.g. >150 RMP), and low spray nozzle pressure (e.g. <1.5 Bar).

In any embodiment, the oil is sprayed into the fluidised bed chamber at low flow rate (e.g. 18-22 RPM), high airflow (e.g. >150 RMP), and low spray nozzle pressure (e.g. <1.5 Bar).

In any embodiment, the suspension of denatured plant protein is sprayed into the fluidised bed drying chamber at high flow rate (e.g. 23-30 RPM), high airflow (e.g. >150 RMP), and high spray nozzle pressure (e.g. >2.5 Bar).

In any embodiment, the API is added in the form of an oil-in-water nanoemulsion, in which the API is contained in the oil phase of the oil-in-water nanoemulsion.

In any embodiment, the oil-in-water nanoemulsion is an oil-in-water nanoemulsion of the invention.

In any embodiment, the carrier material is native protein typically having a moisture content of about or less than 5%, 4%, 3%, or 2%.

In any embodiment, plant the oil is an edible oil, such as a high oleic oil.

In any embodiment, the bed is fluidised with air at 30 to 40° C. and a high airflow rate (approximately 30 to 55 RPM).

In any embodiment, the native protein and denatured protein are plant protein, for example pea or mung bean protein.

In any embodiment, the native protein and denatured protein are protein isolates.

In any embodiment, the suspension of denatured plant protein is a denatured plant protein suspension of the invention.

In any embodiment, the agglomerated microparticles are gastric resistant and ileal sensitive. This means that the microparticles can pass through a human stomach intact and break up in the ileum releasing the API payload.

In another aspect, the invention provides a particulate composition, or a microparticle, obtained by a method of the invention. The particles in the composition may be microparticles. The particles/microparticles may be microbeads, microcapsules, agglomerates, granules or other types of particles. Typically, the composition is a powder. Typically, the microparticles are dried.

In another aspect, the invention provides a microparticle comprising API contained within a optionally polymerised denatured plant protein structure.

In any embodiment, the API is contained within a carrier. The carrier may be edible oil droplets, in which the API is contained within the edible droplets, or a carrier powder such as native protein or starch.

In any embodiment, the polymerised denatured plant protein structure is a polymerised denatured plant protein matrix, and the oil droplets are distributed through the matrix.

In any embodiment, the microparticle has a core-shell structure comprising a denatured plant protein shell and a core comprising the API in a carrier. The carrier may be a powder (as described above), an oil-in-water nanoemulsion of the invention, edible oil droplets, or a solution/suspension of API.

In any embodiment, the drops of edible oil in the nanoemulsion have an average dimension of 20 to 60 nm.

In any embodiment, the denatured plant protein shell is polymerised.

In any embodiment, the microparticle has two or more shells in which at least one shell is a denatured plant protein shell and at least one shell is selected from an oil shell, and a non-polymerised denatured plant protein shell.

In any embodiment, the microparticle is a granule.

In another aspect, the invention provides a pharmaceutical composition comprising microparticles of the invention in combination with a suitable pharmaceutical excipient.

In any embodiment, the pharmaceutical composition is an oral dosage form

In any embodiment, the pharmaceutical composition is provided as a unit dose comprising 10-100 IU of a hydrophobic drug.

In any embodiment, the unit dose comprises 10-50 IU of a hydrophobic drug.

In any embodiment, at least 90% of the hydrophobic drug in the oral dosage form is active.

In any embodiment, the hydrophobic drug is insulin.

In any embodiment, the pharmaceutical composition is a vaccine composition and the API is an immunogen or a nucleic acid encoding an immunogen.

In any embodiment, the vaccine composition comprises an adjuvant.

In any embodiment, the adjuvant is contained within a shell of the microparticle or a core of the microparticle.

In any embodiment, the immunogen is selected from a protein, polypeptide, protein subunit, peptide, protein receptor, antigen, and cell extract (typically a cell wall extract or an outer membrane protein (OmP).

Other aspects and preferred embodiments of the invention are defined and described in the other claims set out below.

### BRIEF DESCRIPTION OF THE FIGURES

FIG. 1: Standard curve for detection of GLP-1/Incretin mimetic in plant protein micro-beads, made by double nozzle extrusion encapsulation technique.

FIG. 2: Detection of encapsulated GLP-1/Incretin mimetic in pea protein microbeads using HPLC, made by double nozzle extrusion encapsulation technique.

FIG. 3A: Detection of pea protein microbead agglomerates and digested contents after human digestion (i.e. release in ileum digesta), made by double nozzle extrusion encapsulation technique.

FIG. 3B. Detection and differentiation of GLP-1/Incretin mimetic (22.5 minutes retention time) released from plant protein microbeads after human digestion (i.e. intestinal release in ileum digesta), made by double nozzle extrusion encapsulation technique.

FIG. 4. Detection of GLP-1/Incretin mimetic (retention time 21.5 minutes) after full human digestion-brown, blue pink and black line). Verification of controlled release of APIs. Data also available to show buffer assessment after encapsulation process, which verifies no loss of the active compound during the encapsulation process (data similar to brown line made by single nozzle extrusion encapsulation technique.

FIG. 5. Detection of GLP-1 (A) and an Incretin mimetic (B) using HPLC quantification and Area Under the Curve (AOC) calculations and standard curve verifications using concentration in the range of 0.2-4.5 mg/ml made by double nozzle extrusion encapsulation technique.

FIG. 6. Microscope images of single nozzle (A) and concentric (double) nozzle (B) microcapsules loaded with mRNA. Encapsulation efficiency is calculated to be greater than 75% for single and concentric nozzle processes.

FIG. 7. Microscope images of stabilised mRNA (A) and API (liraglutide) nano-encapsulated emulsions (B). Average nano-particle size is 25-60 nm. made by double nozzle extrusion encapsulation technique.

FIG. 8. Microscope images of a nano- and micro-encapsulated vaccine in mung bean plant protein microcapsules with an average particle size is 455-510 nm. Micro-capsules were made by double nozzle extrusion encapsulation technique.

FIG. 9: Schematic representing the micro- and nano-encapsulation technology utilised for (A) nucleic acids/mRNA using double nozzle extrusion. The microparticles have a core-shell morphology comprising a core of oil-in-water nanoemulsion (blue background) surrounded by a denatured plant protein shell. Black represents the outer edge of the denatured protein shell where the denatured plant protein is heavily polymerised and grey represents the inner part of the denatured protein shell which has a lower degree of polymerisation compared to the outer edge. Green represents a natural permeation enhancer located in the shell, and red represents nanodroplets of oil containing the API/mRNA/immunogen. FIG. 9B illustrates the same system fortified with an Ig (yellow) (Immunoglobulin; Human Serum source) encapsulated by double nozzle extrusion

FIG. 10: Percent TEER expressed as a function of time and mRNA release. Different treatments are presented with different lines: Control (dextran molecule; Diamond Line); Free Nucleic Acid (Square Line); Encapsulated Nucleic Acid jejunal release (Triangle Line); Encapsulated Micro-Nucleic Acid ileal release (circled Line). Micro-capsules were made by double nozzle extrusion encapsulation technique.

FIG. 11. Calculation of Apparent Permeability (Papp) of free, encapsulated nucleic Acids from jejunal and duodenal instillations (n=2). Micro-capsules were made using double nozzle extrusion encapsulation technique.

FIG. 12 Detection of mRNA on basolateral side after 180 minute (n=2). This data verifies mRNA delivery in a nano- and micro-encapsulation format to the basolateral side. Micro-capsules were made using double nozzle extrusion encapsulation technique.

FIG. 13 No detection of mRNA (free nucleic acid samples) on basolateral side after 180 minutes (n=2). This data verifies absence of mRNA on basolateral side after digestion.

FIG. 14. HPLC evaluation of (A) native, (B) denatured pea protein without permeation enhancers and (C) denatured pea protein with permeation enhancers.

FIG. 15. (A) Pharmacodynamic of encapsulated liraglutide. Illustration of Cross over design study (3 days plus placebo). (B) Utilisation of a standard glucose tolerance test post 2 hours after microcapsules consumption on each day. (C) A continuous glucose monitoring device was utilised to capture glucose concentrations. Data represents nine oral glucose tolerance tests (3 subjects×3 days). Micro-capsules were made using double nozzle extrusion encapsulation technique using denatured mung bean protein.

FIG. 16. Microscope image of plant protein microcapsules with encapsulated API/incretin hormone. Micro-capsules were made using double nozzle extrusion encapsulation technique with pea protein.

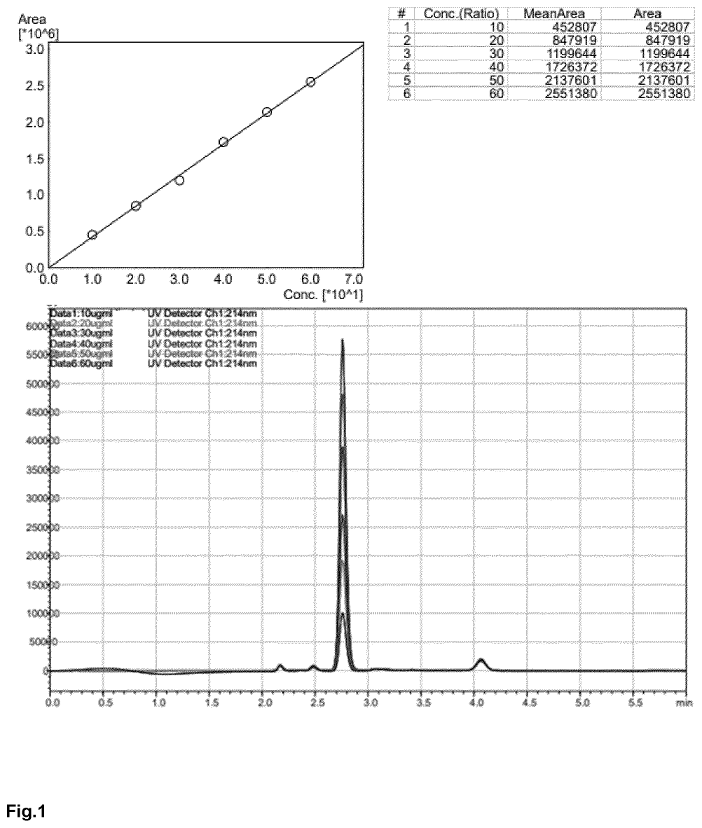

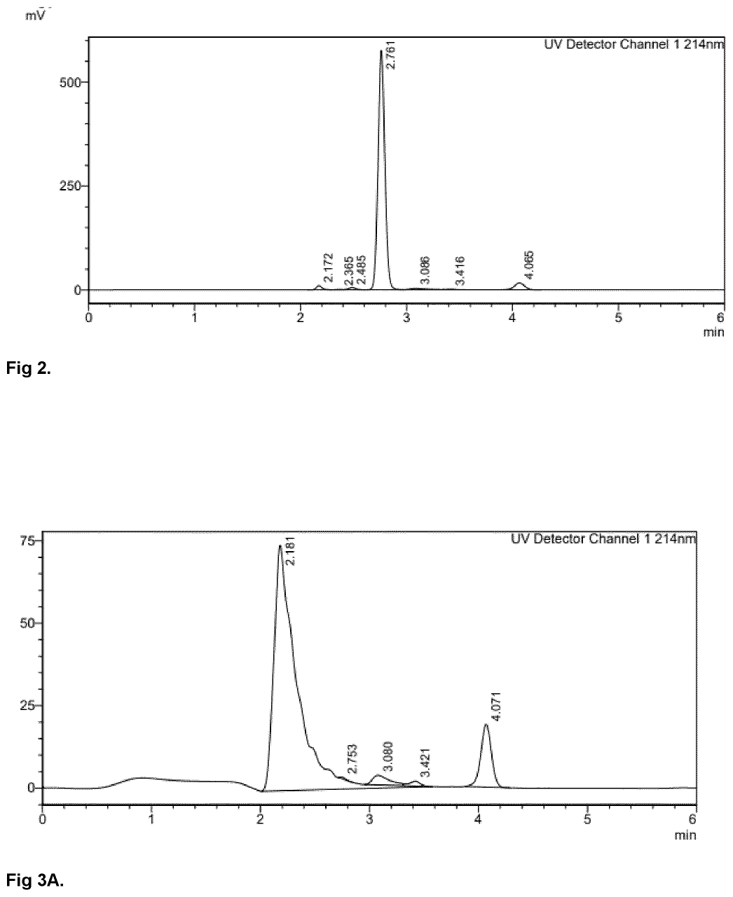

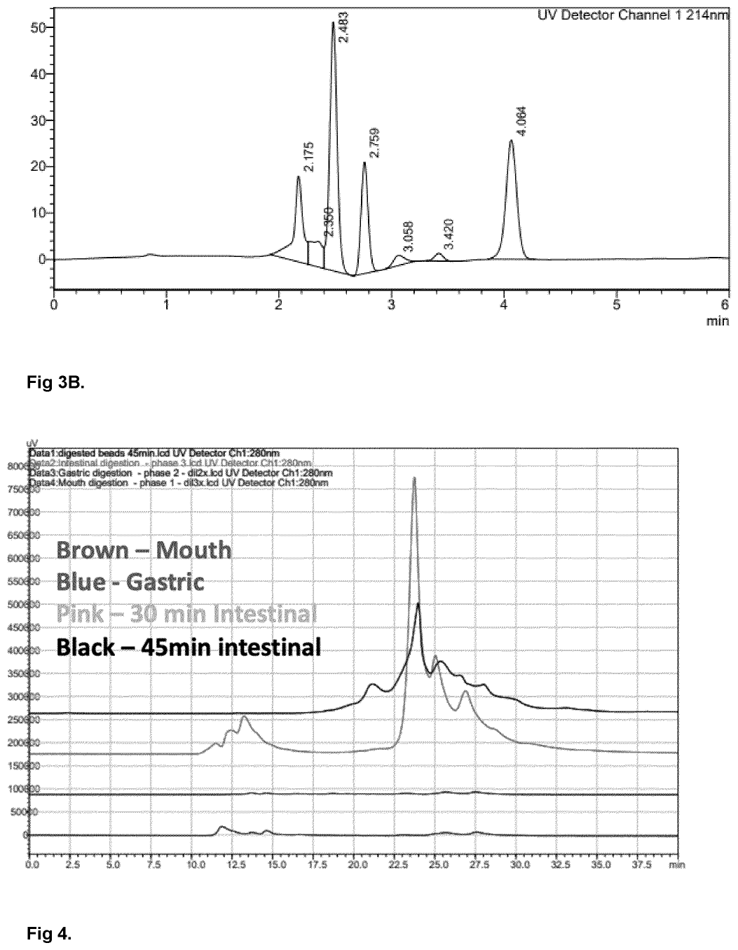

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps. As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological/molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep;

ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

In any embodiment, the microparticles may have an average dimension Dv of (in microns, μm) 50-500, 50-400, 50-300, 50-500, 50-600, 200-700, 50-200, 50-150, 50-100, 20-100, 20-50, 100-500, 100-400, 100-300, or 100-200. When formed by extrusion into a bath, the microparticles typically have an average dimension Dv of 500 to 600 μm. When a single nozzle is employed, the microparticles have a continuous denatured protein matrix and probiotic bacteria distributed throughout the matrix (these are term microbeads). When a concentric nozzle is employed, the microparticles have a core-shell morphology, with a shell comprising denatured protein and a core comprising probiotic bacteria (these are term microcapsules). When formed by gel immobilisation, the microparticle typically have an average dimension Dv of 50 to 150 μm. When formed by spray englobing, the microparticle typically have an average dimension Dv of 50 to 500 μm or 200 to 400 μm.

As used herein, the term “active pharmaceutical ingredient” or “API” refers to a hydrophobic or hydrophilic drug including insulin, liraglutide, a peptide drug, an antigen, a hormone such as an incretin, or an immunogen that may be used in vaccine therapy such as a protein, protein subunit, polypeptide, botanical (e.g. a plant extract) a cell (or a fraction or part of a cell) such as a bacterial cell, viral cell, or yeast cell (e.g. a cell wall fraction), an amino acid or a nucleic acid encoding an immunogen. The nucleic acid may be or comprise an RNA (e.g. mRNA) or DNA molecule. The peptide may be a mitochondrial-targeted peptide.

In this specification, the term “vaccine” should be understood to mean a composition comprising at least an immunogen (e.g. an immunogenic peptide, protein, or antigen) or a nucleic acid encoding an immunogen, and optionally a suitable adjuvant and/or carrier. The vaccine of this invention may be an enteric vaccine or a respiratory vaccine. Examples of immunogens employed in enteric vaccines include an antigen (or nucleic acid that encodes an antigen) configured to generate an immune response against a pathogen selected from S. Aureus, Klebsiella Pneumoniae, Pseudomonas Aeruginosa, Cytomegalovirus, F. Tularensis, HPV, C. Difficile, Shigella, VTEC, norovirus, Salmonella, V. Cholera, Campylobacter, H. Pylori, B. pseudomallei and Giardia. Examples of immunogens employed in respiratory vaccines include, but are not limited, an antigen (or nucleic acid that encodes an antigen) configured to generate an immune response against a pathogen selected from to Cryptosporidium and Pseudomonas Aeruginosa. The antigen may be a protein, protein subunit, cell fraction or inactivated cell. The preparation of vaccines comprising peptide or proteins as active agent is well described in the literature, for example U.S. Pat. Nos. 4,599,230 and 4,601,903, the complete contents of which are incorporated herein by reference. The nucleic acid may be provided in the form of a nucleic acid construct capable of being delivered to a patient and expressing the encoded immunogen in the patient. The nucleic acid construct is preferably in the form of an expression vector, the detail of which will be known to those skilled in the art, for example a plasmid or a virus such as a lentivirus. DNA vaccines are discussed in detail in Kutzler et al [28]. mRNA vaccines are described in Kowalski P S, Rudra A, Miao L, Anderson D G (April 2019). “Delivering the Messenger: Advances in Technologies for Therapeutic mRNA Delivery”. Mol Ther. 27 (4): 710-28; and Verbeke R, Lentacker I, De Smedt S C, Dewitte H (October 2019). “Three decades of messenger RNA vaccine development”. Nano Today. 28:100766.

In this specification, the term “vaccine therapy” should be understood to mean the administration of a vaccine to a mammal with a view to eliciting a response by the host immune system that generally results in the immunogen being destroyed and/or subsequently recognised by the host immune system.

In this specification, the term “adjuvant” should be understood to mean an agent that enhances the recipient's immune response to an immunogen. Adjuvants are included in vaccine formulations to enhance the efficacy of weak antigens and/or to induce appropriate immune responses not sufficiently induced in the absence of the adjuvant. Adjuvants currently employed in vaccines licensed for use in US and Europe include, aluminium salts, oil-in-water emulsions (MF59, AS03, and AF03), virosomes and AS04 (monophosphoryl lipid A preparation (MPL) with aluminium salt). Adjuvant and formulation selection may be based on several parameters, including the physical and chemical natures of the vaccine antigen, type of immune response desired, age of the target population and route of vaccine administration.

As used herein the term “hydrophobic drug” refers to an active biopharmaceutical ingredient that is classed as a Class 3 or 4 under the Biopharmaceutical Classification System (BCS) published by the FDA December 2017. Examples include peptide therapeutics such as insulin, desmopressin, octreotide, cyclosporin, vanomycin, salmon calcitonin, semaglutide, exenatide and insulin or an insulin analogue. Insulin may be insulin degludec or insulin aspart.

As used herein, the term “oral dose form” refers to a pharmaceutical composition formulated for oral administration. Examples include tablets, pills, capsules, thin films, pastes, gels, powders, granules, liquid solutions or suspension. Tablets may be formed by direct compression. The oral dosage form generally includes an API and a pharmaceutical excipient. In one aspect of the present invention, the API is provided in the form of a microcapsule, in which the API is contained within a matrix configured to protect the API during gastric transit and release the API in the ileum. To this end, the matrix may comprise gelated denatured plant protein such as pea protein. In one embodiment the oral dosage form is provided as a unit dose, containing a single dose of API. The API is generally selected from a peptide, protein, antigen, nucleic acid and a hydrophobic or hydrophilic drug.

As used herein, the term “plant protein” refers to a protein preparation obtained from a plant source. Examples include plant protein powders, plant protein concentrates and plant protein isolates. Plant protein preparations can be obtained from pea, chickpea, mung bean, soy, lentil, quinoa and other plant sources. Plant proteins having an isoelectric point above pH 5, 5.5 or 6 are preferred. Typically, the denatured plant protein has a degree of denaturation of at least 60%, 70%, 80%, 90% or 95%. Generally, the method involves an initial step of denaturing the plant protein. Ideally, the plant protein is heat-denatured, although other methods of denaturation are also applicable, for example pressure-induced denaturation. “Pea protein” should be understood to mean a source of pea protein, for example total pea protein. Preferably the pea protein is pea protein isolate (PPI), pea protein concentrate (PPC), or a combination of either. In one embodiment, the pea protein has a purity of at least 70% (i.e. at least 70% by weight of the source of pea protein is pea protein). In one embodiment, the pea protein has a purity of at least 80%. In one embodiment, the pea protein has a purity of at least 90%. Examples of pea protein having a high purity include materials with low ash content.

As used herein, the term “simple carbohydrate” includes monosaccharides such as glucose, fructose and sucrose and disaccharides such as sucrose, lactose and maltose, maltodextrin, and mixtures thereof. In one embodiment, the simple carbohydrate is or comprises Glucidex.

As used herein, the term “calcium salt” refers to a salt of calcium, for example calcium citrate or calcium chloride. The term chelating salt refers to a calcium or magnesium salt configured to polymerise denatured plant protein.

“Nozzle extrusion”: A preferred method of producing the microbeads is a vibrating nozzle technique, in which the suspension is sprayed (extruded) through a nozzle and laminar break-up of the sprayed jet is induced by applying a sinusoidal frequency with defined amplitude to the spray from the nozzle. Examples of vibrating nozzle machines are the Encapsulator (Inotech, Switzerland) and a machine produced by Nisco Engineering AG, or equivalent scale-up version such as those produced by Brace GmbH. Typically, the spray nozzle has an aperture of between 50 and 600 microns, preferably between 50 and 200 microns, suitably 50-200 microns, typically 50-150 microns, and ideally about 80-150 microns. Suitably, the frequency of operation of the vibrating nozzle is from 900 to 3000 Hz. Generally, the electrostatic potential between nozzle and acidification bath is 0.85 to 1.3 V. Suitably, the amplitude is from 4.7 kV to 7 kV. Typically, the falling distance (from the nozzle to the acidification bath) is less than 50 cm, preferably less than 40 cm, suitably between 20 and 40 cm, preferably between 25 and 35 cm, and ideally about 30 cm. The flow rate of suspension (passing through the nozzle) is typically from 3.0 to 10 ml/min; an ideal flow rate is dependent upon the nozzle size utilized within the process.

“Vacuum drying” is the mass transfer operation in which the moisture present in a substance, usually a wet solid, is removed by means of creating a vacuum. In chemical processing industries like food processing, pharmacology, agriculture, and textiles, drying is an essential unit operation to remove moisture. Vacuum drying is generally used for the drying of substances which are hygroscopic and heat sensitive, and is based on the principle of creating a vacuum to decrease the chamber pressure below the vapor pressure of the water, causing it to boil. With the help of vacuum pumps, the pressure is reduced around the substance to be dried. This decreases the boiling point of water inside that product and thereby increases the rate of evaporation significantly. The result is a significantly increased drying rate of the product. The pressure maintained in vacuum drying is generally 0.03-0.06 atm and the boiling point of water is 25-30° C. The vacuum drying process is a batch operation performed at reduced pressures and lower relative humidity compared to ambient pressure, enabling faster drying. “Water activity” (aw) is the partial vapor pressure of water in a substance divided by the standard state partial vapor pressure of water. It is measured by the method described in Carter, B. P., Galloway, M. T., Campbell, G. S., & Carter, A. H. (2015). The critical water activity from dynamic dewpoint isotherms as an indicator of pre-mix powder stability. Journal of Food Measurement and Characterization, 9 (4), 479-486. The operator's manual of the equipment used is provided at http://manuals.decagon.com/Manuals/13893_AquaLab%20Pre_Web.pdf Values for water activity (Aw) provided herein are obtained at 25° C. unless stated otherwise.

“Fluidised air drying” refers to an air-drying approach to control the gentle and homogenous drying of wet solids such as micro-capsules. The intensive mass exchange of air fluidized between micro-capsules makes this method effective and suitable for post-drying of micro-capsules and agglomerated micro-capsules.

The disclosure presents a natural, micro-encapsulated protein microparticle suitable for delivery intact to the mammalian lower intestine via an oral route and comprising a matrix formed from optionally polymerised denatured plant protein, typically with a natural permeation enhancer present to support the absorption kinetics. This natural micro-encapsulation technology provides many technical solutions for antiemetics, antihistamines, and peptide hormones such as Incretin mimetics, and related Active Pharmaceutical Ingredients (APIs), vaccines, mycoplasma and protein/peptide of various sizes.

Plant protein is used as an encapsulation matrix in microparticles because of its capacity for transition congealing. These gel structures can be formed by cold-set congealing, which does not subject the cargo to harsh temperatures or solvents, making it ideal for various therapeutics, vaccines and incretin mimetics for micro-encapsulation. The generation of a nano-encapsulation system further stabilise the active compound during the encapsulation process and provide a mechanism for permeation into cytosol of the mammal.

Transition congealing requires a dual process (solubilisation+thermal), to provide the structural arrangement necessary to form micro-capsules via in the presence of divalent salts (ionic strength and pH). Generally, the microcapsules have a micron-sized dimension (e.g. an average dimension of 30-50 microns). The microcapsules can be a mononuclear morphology where the API is provided by a single core contained within a plant protein shell or may be multinuclear in which discrete pockets of API are homogenously distributed throughout a plant protein matrix. The protein matrix of the microcapsules is gastric resistant and susceptible for break-up in the ileum, thus enabling delivery of API's through the acidic stomach conditions intact for release in the proximal ileum. The nano-encapsulation structure provides the vehicle to allow the API to bioavailable for transport into cell cytosol; which is based on a charge interaction.

Benefits of the combined nano- and micro-encapsulation technology include one or more of the following:
Stabilisation of API/antigen/incretin mimetic/protein for GI delivery and nano-encapsulation for activated cell penetration
Delivery Efficacy for improved API stability and efficacy against Rnase degradation (for mRNA) and protein digestion (peptides) whilst transiting through the human body with efficient diffusion across cell membranes.
Targeted delivery to site of action due to natural digestion mechanism of plant protein excipients.
Natural biodegradability using a food-grade encapsulation platform.
Inclusion of permeation enhancers which accelerates the transport of therapeutics and nucleotides across the intestinal mucosa for possible enhanced immunity.
Delivery of APIs in micro-capsule form which circumvents the necessity of dealing with liquid dosage forms, which eliminates RNase contamination risk.
Reduced supply chain and distribution costs relative to current injection options.
Removal of sterilization requirements related to injectables and reduced healthcare costs by eliminating the need for trained professionals to administer certain dosage forms.

Preliminary trials conducted indicate that the combination of micro- and nano-encapsulation support the delivery of mRNA vaccines and Incretin memetics through human digestion (without degradation) for enhanced uptake into the blood, leading to “active” vaccine uptake into the cell. This natural encapsulation technology has the potential to deliver vaccines and APIs orally with possible enhanced immune responses without significant losses or damage to the active along this journey.

In the context of vaccinating a global population the benefits of delivering a vaccine orally are immense: notwithstanding the cost benefits, an oral treatment would make it far easier to reach people in developing countries where supply chain and in particular cold chain challenges are greatest. Delivery of vaccines in a tablet format circumvents the need for liquid injectables, which eliminates contamination risk and reduces healthcare costs relating to administration of the injection. I

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1

### Preparation of Denatured Pea Protein

Hydrate 14% (w/v) pea protein isolate in water or phosphate buffer 0.1M pH7.5
Hydrate at 30° C. for 45 minutes
Agitate at 450 RPM using jacketed vessel or hot plate at 30° C.
Leave for minimum 90 min as the request resting step
Check pH of water and verify pH 7.5—if not, adjust using 1 N HCl/NaOH
Heat treat as follows:
   Pre heat 65° C. for a minimum of 5 seconds/maximum of 15 minutes
   Final heat 95.5° C. for a minimum of 2 seconds/maximum of 3 minutes
   Cooling temperature 22-25° C.
   Flow Rate 1.5 L-2.0 L per minute
   Pressure 1.3-1.6 bar
Allow material to settle for 4 hour at room temperature and it is ready for use
This material is referred to as “dPPI”.

### Example 2: Ultra Denaturation of Pea Protein

### Preparation of Ultra-Denatured Pea Protein

Hydrate 14% (w/v) pea protein isolate in water or phosphate buffer 0.1M pH 7.5
Hydrate at 30° C. for 45 minutes
Agitate at 450 RPM using jacketed vessel
Leave for minimum 90 minimum as the request resting step
Check pH pf water and verify pH 7.5—if not, adjust using 1 N HCl/NaOH
Heat treat as follows:
Pre heat 60-98° C. for a minimum of 2 seconds
Final heat 126-144.5° C.
Hold time minimum 2 seconds
Cooling temperature 22-25° C.
Flow Rate: min 1.45 L-2.0 L per minute
Pressure 1.0-6.0 bar
Allow material to settle for 2 hour at room temperature and it is ready for use
This material is referred to as “udPPI”

### Example 3

### Preparation of Ultra-Denatured Mung Bean Protein

Hydrate 10% (w/v) mung protein isolate in water or phosphate buffer 0.1M pH7.5
Hydrate at 30° C. for 90 minutes
Agitate at 450 RPM using jacketed vessel
Leave for minimum 90 minimum as the request resting step
Check pH pf water and verify pH 7.5—if not, adjust using 1 N HCl/NaOH
Heat treat as follows:
Pre heat 60-90° C. for a minimum of 2 seconds
Final heat 120-144° C.
Cooling temperature 22-25° C.
Flow Rate 1.5 L-2.0 L per minute
Pressure 1.0-6.0 bar
Allow material to settle for 2 hour at room temperature and it is ready for use
This material is referred to as “udMBP”.

### Example 4

### Preparation of Ultra-Denatured Plant Protein

Hydrate 10% (w/v) plant protein isolate in water or phosphate buffer 0.1M pH7.5
Hydrate at 30° C. for 45 minutes
Agitate at 450 RPM using jacketed vessel
Leave for minimum 90 min as the request resting step
Check pH pf water and verify pH 7.5—if not, adjust using 1 N HCl/NaOH
Heat treat as follows:
Pre heat 60-90° C. for a minimum of 2 seconds
Final heat 120-144° C.
Cooling temperature 22-25° C.
Flow Rate 1.5 L-2.0 L per minute
Pressure 1.0-6.0 bar
Allow material to settle for 2 hour at room temperature and it is ready for use
This material is referred to as “udPP”

### Example 5

Encapsulation of mRNA Nano-Emulsion in Microbeads Using Denatured PPI (Single Nozzle Extruder)
Agitate dPPI prepared as per example 1 for 1 hour min at 250 RPM at RT
Prepare RNA suspension Oil in water emulsion (See Table 1).

TABLE 1

| | Formulation A | Formulation B |
|---|---|---|
| Composition | (with IgA) | (no IgA) |
| RNA | 80% | 80% |
| High Oleic Oil | 5% | 7.5% |
| Immunoglobulin A (IgA) | 5% | N/A |
| dPPI | 7.5% | 10% |
| Tween 80 | 2.5% | 2.5% |

Ingredients of Table 1 (solids) are added to water in a solids: water weight ratio of 1:1.
Emulsify at 5,000 RPM for 45 seconds
Allow the material to settle for 10 minutes
Prepare Calcium buffer at pH 6.1; 0.4M concentration
Add RNA suspension of Table 1 to dPPI solution dropwise in 9:1 ratio (protein: RNA suspension)
Agitate at 450 RPM for 5 minutes min
Extrude through a single nozzle and polymerize in Calcium buffer at pH 6.1
Microcapsules are collected, washed and equilibrated to pH 6.5 before drying
Microcapsules are loaded to the relevant drying chamber
Drying can be conducted as follows:
   Freeze Drying (See Table 1); or
   Fluidised Bed Drying: 37° C. for 4-6 Hours; or
   Vacuum Drying: Less than 10 mBar for 24 hours min at 37° C.

### Example 6

Encapsulation of mRNA Nano-Emulsion in Microbeads Using Denatured PPI (Concentric Nozzle Extruder)

Microcapsules are prepared as per Example 5 with the exception that the RNA suspension of Table 1 is extruded through the inner nozzle of the extruder and the dPPI of Example 1 is simultaneously extruded through the outer nozzle. In this example, 2% glucidex/glucose/simple carbohydrate is added to the dPPI prior to encapsulation (this adds charge to the denatured protein solution and creates a compatible matrix for probiotics)

### Example 7

### Encapsulation of Insulin Nano-Emulsion in Microbeads Using Denatured PPI (Single Nozzle Extruder)

Agitate dPPI prepared as per Example 1 for 1 hour minimum at 250 RPM at RT
Prepare Insulin suspension Oil in water emulsion (See Table 2).

TABLE 2

| Composition | Formulation |
|---|---|
| Insulin | 80% |
| High Oleic Oil | 4% |
| dPPI | 15.5% |
| Tween 80 | 1.5% |

Ingredients of Table 2 (solids) are added to water in a solids: water weight ratio of 1:1.
Emulsify at 5,000 RPM for 45 seconds
Allow the material to settle for 10 minutes
Prepare Calcium citrate buffer at pH 5.5-6.1; 0.1M concentration
Add Insulin suspension (o/w emulsion) of Table 2 to dPPI solution dropwise in 9:1 ratio (protein: API suspension)
Agitate at 450 RPM for 5 minutes minimum
Homogenise at 18,000 RPM for 30 seconds-repeat for 2 cycles
Allow to settle for 20 minutes at 4° C.
Extrude Insulin Oil emulsion through a single nozzle and polymerize in Calcium citrate buffer at pH 6.1 (0.1M)
Microcapsules are collected, washed and equilibrated to pH 6.5 before drying
Microcapsules are loaded to the relevant drying chamber
Drying can be conducted as follows:
Freeze Drying (See Table 1); or
Fluidised Bed Drying: 37° C. for 4-6 Hours; or
Vacuum Drying: Less than 10 mBar for 24 hours minimum at 37° C.

### Example 8

### Encapsulation of Insulin Nano-Emulsion in Microbeads Using Denatured PPI (Double Nozzle Extruder)

Microcapsules are prepared as per Example 7 with the exception that the Insulin suspension (o/w emulsion) of Table 2 is extruded through the inner nozzle of the extruder and the dPPI of Example 1 is simultaneously extruded through the outer nozzle. In this example, 2% glucidex/glucose/simple carbohydrate is added to the dPPI prior to encapsulation (this adds charge to the denatured protein solution the creates a compatible matrix for an API).

### Example 9

Spray Englobing of mRNA in Denatured PPI
Agitate dPPI prepared as per Example 1 for 1 hour minimum at 250 RPM at RT
Prepare RNA suspension Oil in water emulsion (See Table 1).
Add 2% glucidex/glucose/simple carbohydrate to the dPPI
Hydrate at room temperature for 2 hours using mild agitation to avoid creation of air pockets
Load fluidised bed chamber with dry pea protein powder (native powder; approx. 2% moisture).
Fluidise at 37° C. at high airflow
Added RNA suspension Oil in water emulsion to the powder at 1:5 or 1:10 ratio
Add 15% (by weight of solids) Calcium Citrate to the chamber and fluidise
Spray 10% glucidex solution (1-12%) onto the material at high flow rate (20-30 RPM) high airflow (>150 RPM) and low nozzle pressure (<1.5 bar) promotes granulation
Spray coconut oil/sunflower oil/high oleic oil onto the powder at low flow rate (18-22 RPM), high airflow (>150 RPM) and high nozzle pressure (>2.5 bar); promotes englobing
Spray dPPI—glucidex solution into chamber at high flow rate (25-30 RPM), high airflow (>150 RPM) and high nozzle pressure (>2.5 bar); promotes englobing.
CalciumCitrate in the drying chamber will act as a Calcium-chelate and polymerise the dPPI to enhance coating and entrapment of o/w emulsion in the powder.
Post dry the material to achieve Aw<0.15.

### Example 10

Immobilisation of mRNA Nanoemulsion in Denatured PPI Made as Per Example 1
Preparation of mRNA Nano-Emulsion
Prepare RNA suspension Oil in water emulsion as per Example 5 and Table 1.
Prepare Calcium Citrate buffer at pH 6.1; 0.1M concentration
Using dPPI prepared as per Example 1, prepare a suspension of PPI
Add 2% glucidex/glucose/simple carbohydrate to the protein solution prior to encapsulation (this adds charge to the denatured protein solution the create a compatible matrix for APIs).
Hydrate at RT for 2 hours using mild agitation to avoid creation of air pockets
Equilibrate mRNA to room temperature and after 2 minutes add to dPPI
Add RNA suspension Oil in water emulsion to dPPI solution dropwise in 9:1 ratio (protein: RNA suspension)
Agitate at 450 RPM for 5 minutes minimum
After addition of mRNA, add 0.1M/pH 6.1 Calcium Citrate
Add the Calcium Citrate until the buffer/suspension ratio is 1:300 or 1:500.
During pH adjustment, maintain high level of agitation at 450 RPM RT
Maintain agitation for 15 minutes minimum and transfer to drying chamber
The solution will visibly appear more viscous.
Drying can be conducted by freeze drying or Vacuum drying as follows Freeze Drying (See Table 3)

TABLE 3

| | |
|---|---|
| Temperature | Time |

| | | | |
|---|---|---|---|
| −25° | C. | 23 | Hours |
| −10° | C. | 25 | Hours |
| 0° | C. | 8 | Hours |
| 10° | C. | 8 | Hours |
| 20° | C. | 4 | Hours |
| 25° | C. | 4 | Hours |

Total Drying Time = 72 Hours

Vacuum Drying:
Set product temperature to 25° C.
Create vacuum in product chamber
Dry for 24H once full vacuum is achieved i.e. <10 mBar

### Example 11: Encapsulation of VTEC/Single Nozzle/Denatured Pea Protein

Encapsulation of Verocytotoxin-Producing E. coli (VTEC)/Zoonotic Pathogen Via the Generation of Nano-Emulsions Entrapped in Microbeads Using Denatured Pea Protein (Single Nozzle Extruder)
Agitate dPPI prepared as per example 1 for 1 hour minimum at 250 RPM at RT
Prepare VTEC suspension Oil in water emulsion (See Table 4).

TABLE 4

| | Formulation A | Formulation B |
|---|---|---|
| Composition | (with IgA) | (no IgA) |
| VTEC | 80% | 80% |
| Sunflower oil | 5% | 7.5% |
| Immunoglobulin | 5% | N/A |
| Denatured Pea Protein | 7.5% | 10% |
| Tween 20 or 80 | 2.5% | 2.5% |

Ingredients of Table 4 (solids) are added to water in a solids: water weight ratio of 1:1.
Emulsify at 5,000 RPM for 45 seconds
Allow the material to settle for 10 minutes
Prepare Calcium buffer at pH 6.1; 0.4M-0.6M concentration
Add VTEC suspension of Table 1 to denatured protein solution dropwise in 9:1 ratio (protein: VTEC suspension)
Agitate at 450 RPM for 5 minutes minimum
Extrude through a single nozzle and polymerize in Calcium buffer at pH 6.1
Microcapsules are collected, washed and equilibrated to pH 6.5 before drying
Microcapsules are loaded to the relevant drying chamber
Drying can be conducted as follows:
   Freeze Drying (See Table 3); or
   Fluidised Bed Drying: 37° C. for 4-6 Hours; or
   Single stage Vacuum Drying: <10 mBar for 24 hours minimum at 37° C.
   2-stage Vacuum Drying: <10 mBar for 24-30 hours minimum at 37° C.

### Example 12: Encapsulation Bacterial Antigen/Denatured Pea Protein/Single Nozzle

### Encapsulation of Bacterial (De-Active, Dead Cells) Protein/Antigen Via the Generation of Nano-Emulsions Entrapped in Microbeads Using Denatured Pea Protein (Single Nozzle Extruder)

Agitate dPPI prepared as per example 1 for 1 hour minimum at 250 RPM at RT
Prepare bacterial suspension Oil in water emulsion (See Table 5).

TABLE 5

| | Formulation A | Formulation B |
|---|---|---|
| Composition | (with IgA) | (no IgA) |
| Bacterial biomass | 80% | 80% |
| Sunflower oil | 5% | 7.5% |
| Immunoglobulin | 5% | N/A |
| Denatured Pea Protein | 7.5% | 10% |
| Tween 20 or 80 | 2.5% | 2.5% |

Ingredients of Table 5 (solids) are added to water in a solids: water weight ratio of 1:1.
Emulsify at 5,000 RPM for 45 seconds
Allow the material to settle for 10 minutes
Prepare Calcium buffer at pH 6.1; 0.4 M-0.6 M concentration
Add bacterial suspension of Table 1 to denatured protein solution dropwise in 9:1 ratio (protein: VTEC suspension)
Agitate at 450 RPM for 5 minutes minimum
Extrude through a single nozzle and polymerize in Calcium buffer at pH 6.1
Microcapsules are collected, washed and equilibrated to pH 6.5 before drying
Microcapsules are loaded to the relevant drying chamber
Drying can be conducted as follows:
   Freeze Drying (See Table 3); or
   Fluidised Bed Drying: 37° C. for 4-6 Hours; or
   Single stage Vacuum Drying: <10 mBar for 24 hours minimum at 37° C.
   2-stage Vacuum Drying: <10 mBar for 24-30 hours minimum at 37° C.

### Example 13: Encapsulation VTEC Antigen/Single Nozzle/Denatured Mung Bean

Encapsulation of Verocytotoxin-Producing E. coli (VTEC Antigen)/Zoonotic Pathogen Via the Generation of Nano-Emulsions Entrapped in Microbeads Using Denatured Mung Bean Protein (Single Nozzle Extruder)
Agitate denatured mung bean protein prepared as per example 3 for 1 hour minimum at 250 RPM at RT
Prepare VTEC suspension Oil in water emulsion (See Table 6).

TABLE 6

| | Formulation A | Formulation B |
|---|---|---|
| Composition | (with IgA) | (no IgA) |
| VTEC | 80% | 80% |
| Sunflower oil | 4.5% | 7.5% |
| Immunoglobulin | 5% | N/A |
| Denatured mung bean | 10% | 10% |
| protein | | |
| Tween 20 or 80 | 0.5% | 2.5% |

Ingredients of Table 6 (solids) are added to water in a solids: water weight ratio of 1:1.
Emulsify at 5,000 RPM for 45 seconds
Allow the material to settle for 10 minutes
Prepare Calcium buffer at pH 6.1; 0.4M-0.6M concentration
Add VTEC suspension of Table 6 to denatured protein solution dropwise in 9:1 ratio (protein: VTEC suspension)
Agitate at 450 RPM for 5 minutes minimum
Extrude through a single nozzle and polymerize in Calcium buffer at pH 6.1
Microcapsules are collected, washed and equilibrated to pH 6.5 before drying
Microcapsules are loaded to the relevant drying chamber
Drying can be conducted as follows:
   Freeze Drying (See Table 3); or
   Fluidised Bed Drying: 37° C. for 4-6 Hours; or
   Single stage Vacuum Drying: <10 mBar for 24 hours min at 37° C.
   2-stage Vacuum Drying: <10 mBar for 24-30 hours min at 37° C.

### Example 14: Encapsulation of Bacterial Antigen/Concentric Nozzle/Denatured Mung Bean

### Encapsulation of Bacterial (De-Active, Dead Cells) Antigen Via the Generation of Nano-Emulsions Entrapped in Microbeads Using Denatured Mung Bean Protein (Double Nozzle Extruder)

Agitate denatured mung bean protein prepared as per example 3 for 1 hour min at 250 RPM at RT
Prepare bacterial suspension Oil in water emulsion (See Table 7).

TABLE 7

| | Formulation A | Formulation B |
|---|---|---|
| Composition | (with IgA) | (no IgA) |
| Bacterial biomass | 80% | 80% |
| Sunflower oil | 4.5% | 7.5% |
| Immunoglobulin | 5% | N/A |
| Denatured mung bean | 10% | 10% |
| protein | | |
| Tween 20 or 80 | 0.5% | 2.5% |

Ingredients of Table 7 (solids) are added to water in a solids: water weight ratio of 1:1.
Emulsify at 5,000 RPM for 45 seconds
Allow the material to settle for 10 minutes
Prepare Calcium buffer at pH 6.1; 0.4M-0.6M concentration
Add bacterial suspension of Table 7 to denatured protein solution dropwise in 9:1 ratio (protein: VTEC suspension)
Agitate at 450 RPM for 5 minutes min
Extrude through a double nozzle and polymerize in Calcium buffer at pH 6.1
Microcapsules are collected, washed and equilibrated to pH 6.5 before drying
Microcapsules are loaded to the relevant drying chamber
Drying can be conducted as follows:
   Freeze Drying (See Table 3); or
   Fluidised Bed Drying: 37° C. for 4-6 Hours; or
   Single stage Vacuum Drying: <10 mBar for 24 hours min at 37° C.
   2-stage Vacuum Drying: <10 mBar for 24-30 hours min at 37° C.

### Example 15: Encapsulation of OMP/Denatured Mung Bean/Concentric Nozzle

### Encapsulation of Outer Membrane Protein (OmP) Antigen Via the Generation of Nano-Emulsions Entrapped in Microbeads Using Denatured Mung Bean Protein (Concentric Nozzle Extruder)

Agitate denatured mung bean protein prepared as per example 3 for 1 hour min at 250 RPM at RT
Prepare outer membrane protein antigen in Oil in water emulsion (See Table 8).

TABLE 8

| | Formulation A | Formulation B |
|---|---|---|
| Composition | (with IgA) | (no IgA) |
| Outer membrane protein | 75% | 80% |
| antigen | | |
| Sunflower oil | 4.5% | 7.5% |
| Immunoglobulin | 5% | N/A |
| Denatured mung bean | 15% | 10% |
| protein | | |
| Tween 20 or 80 | 0.5% | 2.5% |

Ingredients of Table 8 (solids) are added to water in a solids: water weight ratio of 1:1.
Emulsify at 5,000 RPM for 45 seconds
Allow the material to settle for 10 minutes
Prepare Calcium buffer at pH 6.1; 0.4M-0.6M concentration
Add outer membrane protein antigen of Table 8 to denatured protein solution dropwise in 9:1 ratio (protein: VTEC suspension)
Agitate at 450 RPM for 5 minutes min
Extrude through a double nozzle and polymerize in Calcium buffer at pH 6.1
Microcapsules are collected, washed and equilibrated to pH 6.5 before drying
Microcapsules are loaded to the relevant drying chamber
Drying can be conducted as follows:
   Freeze Drying (See Table 3); or
   Fluidised Bed Drying: 37° C. for 4-6 Hours; or
   Single stage Vacuum Drying: <10 mBar for 24 hours min at 37° C.
   2-stage Vacuum Drying: <10 mBar for 24-30 hours min at 37° C.

### Example 16: Encapsulation of OMP+Adjuvant/Denatured Mung Bean/Concentric (Both in the Core)

### Encapsulation of Outer Membrane Protein Antigen and Adjuvant Via the Generation of Nano-Emulsions Entrapped in Microbeads Using Denatured Mung Bean Protein (Double Nozzle Extruder)

Agitate denatured mung bean protein prepared as per example 3 for 1 hour min at 250 RPM at RT
Prepare outer membrane protein antigen in Oil in water emulsion (See Table 9).

TABLE 9

| | Formulation A | Formulation B |
|---|---|---|
| Composition | (with Ig) | (no Ig) |
| Outer membrane protein | 70% | 75% |
| antigen | | |
| Sunflower oil | 4.5% | 7.5% |
| Immunoglobulin | 5% | N/A |
| Adjuvant | 5% | 5% |
| Denatured mung bean | 15% | 10% |
| protein | | |
| Tween 20 or 80 | 0.5% | 2.5% |

Ingredients of Table 9 (solids) are added to water in a solids: water weight ratio of 1:1.
Emulsify at 5,000 RPM for 45 seconds
Allow the material to settle for 10 minutes
Prepare Calcium buffer at pH 6.1; 0.4M-0.6M concentration
Add outer membrane protein antigen of Table 9 to denatured protein solution dropwise in 9:1 ratio (protein: VTEC suspension)
Add adjuvant to inner core with the outer membrane protein (OMP) antigen
Maintain shell material as pure denatured mung bean protein
Agitate at 450 RPM for 5 minutes min
Extrude through a double nozzle (inner core=outer membrane protein antigen+adjuvant; and outer shell=denatured mung bean protein)
Polymerize in Calcium buffer at pH 6.1
Microcapsules are collected, washed and equilibrated to pH 6.5 before drying
Microcapsules are loaded to the relevant drying chamber
Drying can be conducted as follows:
   Freeze Drying (See Table 3); or
   Fluidised Bed Drying: 37° C. for 4-6 Hours; or
   Single stage Vacuum Drying: <10 mBar for 24 hours min at 37° C.
   2-stage Vacuum Drying: <10 mBar for 24-30 hours min at 37° C.

### Example 17: Encapsulation of OMP/Denatured Mung Bean/Concentric (Inner Core Vs Outer Shell)

### Encapsulation of Outer Membrane Protein Antigen and Adjuvant Via the Generation of Nano-Emulsions Entrapped in Microbeads Using Denatured Mung Bean Protein (Double Nozzle Extruder)

Agitate denatured mung bean protein prepared as per example 3 for 1 hour min at 250 RPM at RT
Prepare outer membrane protein antigen in Oil in water emulsion (See Table 10).

TABLE 10

| | Formulation A | Formulation B |
|---|---|---|
| Composition | (with Ig) | (no Ig) |
| Outer membrane protein | 70% | 75% |
| antigen | | |
| Sunflower oil | 4.5% | 7.5% |
| Immunoglobulin | 5% | N/A |
| Adjuvant | 5% | 5% |
| Denatured mung bean | 15% | 10% |
| protein | | |
| Tween 20 or 80 | 0.5% | 2.5% |

Ingredients of Table 10 (solids) are added to water in a solids: water weight ratio of 1:1.
Emulsify at 5,000 RPM for 45 seconds
Allow the material to settle for 10 minutes
Prepare Calcium buffer at pH 6.1; 0.4M-0.6M concentration
Add outer membrane protein (OMP) antigen of Table 10 to denatured protein solution dropwise in 9:1 ratio (protein: VTEC suspension)
Add adjuvant to outer core—combine adjuvant with denatured mung bean protein in outer shell material
Agitate at 450 RPM for 5 minutes min
Extrude through a double nozzle (inner core=outer membrane protein antigen; and outer shell=adjuvant+denatured mung bean protein)
Polymerize in Calcium buffer at pH 6.1
Microcapsules are collected, washed and equilibrated to pH 6.5 before drying
Microcapsules are loaded to the relevant drying chamber
Drying can be conducted as follows:
   Freeze Drying (See Table 3); or
   Fluidised Bed Drying: 37° C. for 4-6 Hours; or
   Single stage Vacuum Drying: <10 mBar for 24 hours min at 37° C.
   2-stage Vacuum Drying: <10 mBar for 24-30 hours min at 37° C.

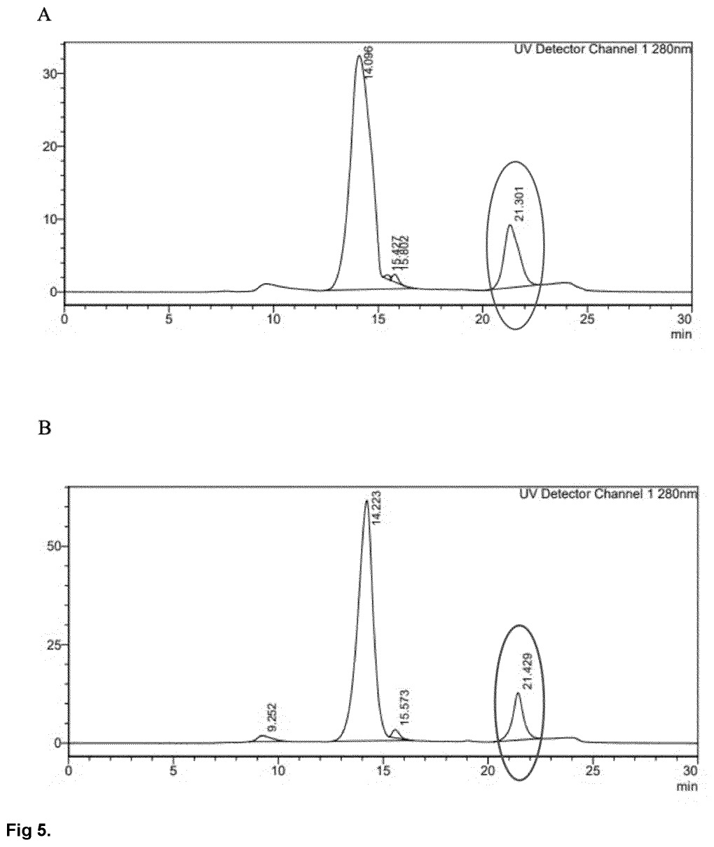

Human in vitro digestion presents the initial phase of this assay to elucidate the stability of encapsulated nucleic acids during stomach and intestinal transit. The procedure consists of subjecting the encapsulated material to a 3-stage digestive process: oral, gastric and small intestinal, with subsequent presentation of the digesta to the intestinal gut issue (method reference: https://www.nature.com/articles/s41596-018-0119-1). FIG. 4 illustrates the controlled release of APIs in the intestinal phase with no/minimal release in mouth or gastric phase. Intestinal liberation of the payload is evident after 30 minutes intestinal incubation. The intestinal digestion process takes place in the Ussing chamber to best mimic the process of permeation and absorption across the gut wall. The concentrations of enzymes and the retention times for each digestion step have been validated and are aligned with the latest research in human digestive physiology. All in vitro solutions are prepared as per World Health Organisation (WHO) and USP standards for determination of bioavailability and tissue permeation.

Percent TEER values are utilized to evaluate permeability of nucleic acid content and their respective bioavailability. A drop in % TEER is directly related to an increase in tissue permeability for the active agent (i.e. nucleic acid) and an increase in mRNA bioavailability on the basolatera (blood side).

↓% Teer=↑Tissue Permeability=↑bioavailability

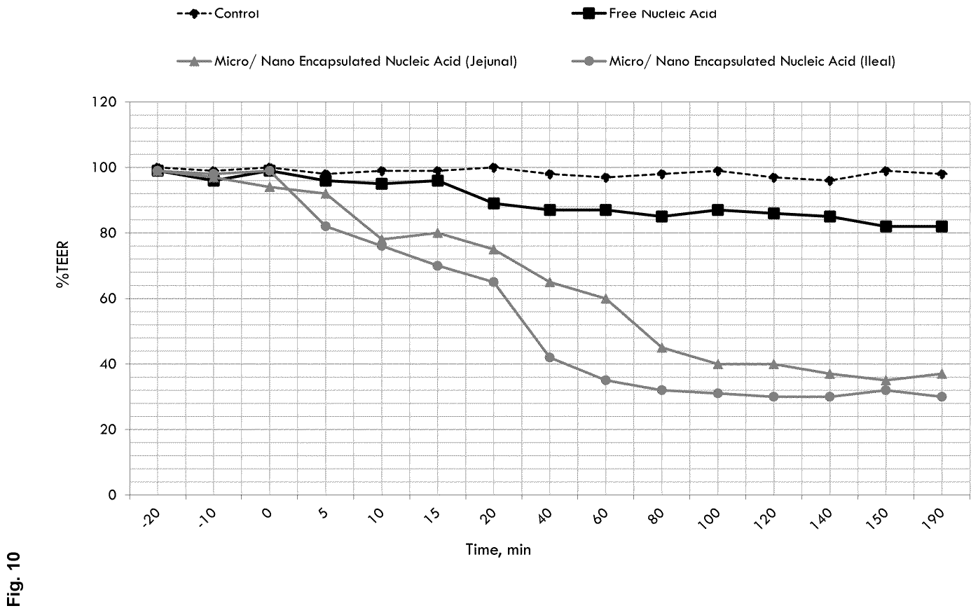

FIG. 10 shows the response for control markers (control; diamond line) exemplifies the readiness of the system for testing. For free nucleic acids (FIG. 10. Square line) has poor permeability across the gut epithelial tissue; illustrating a similar profile to the non-permeable control.

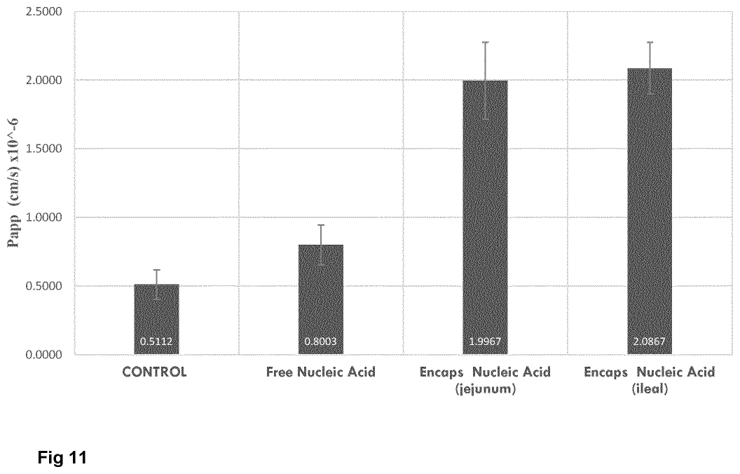

Interestingly, encapsulated nucleic acids (FIG. 10 triangle and circle lines) provide a greater permeability (>50% improved permeation) after 180 minutes, relative to free nucleic acids. FIG. 11 illustrates the significant difference between free nucleic acids relative to encapsulated formats. Apical exposure of the intestinal epithelium to micro-encapsulated nucleic acid did not show any damage or allergic reaction to the mucosa or sub-mucosa (histology not shown in this report). No significant different in Papp values is shown between encapsulated nucleic acids presented to the jejunal or ileal tissues; hence encapsulated materials similar magnitudes of permeation when nucleic acids are exposed to either jejunal or ileal.

TABLE 11
Permeation values after 180 min incubation time

| | P_{APP} ± SEM | STANDARD |
|---|---|---|
| | (CM/S) (×10⁻⁶) | DEVIATION |
| CONTROL | 0.51122 | 0.107 |
| FREE NUCLEIC ACID | 0.8003 | 0.145 |
| ENCAPS NUCLEIC ACID | 1.9967 | 0.280 |
| (JEJUNUM) | | |
| ENCAPS NUCLEIC ACID (ILEUM) | 2.0867 | 0.188 |

This data endorses encapsulation as a chaperon for efficient delivery mRNA without significant loss or tissue damage. It is proposed that encapsulation presents mRNA to the gut wall in a more bioavailable form for relatively fast uptake within 100 minutes of digestion.

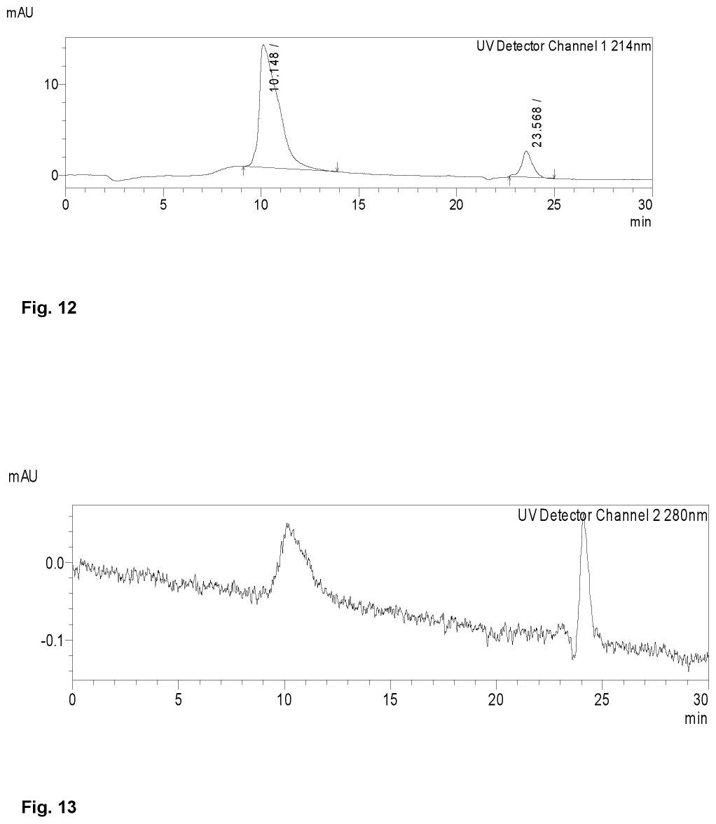

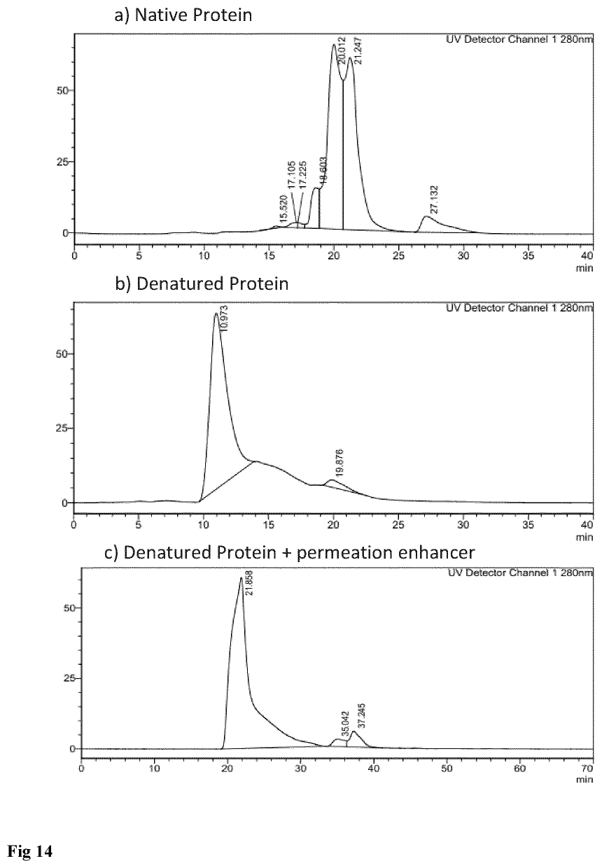

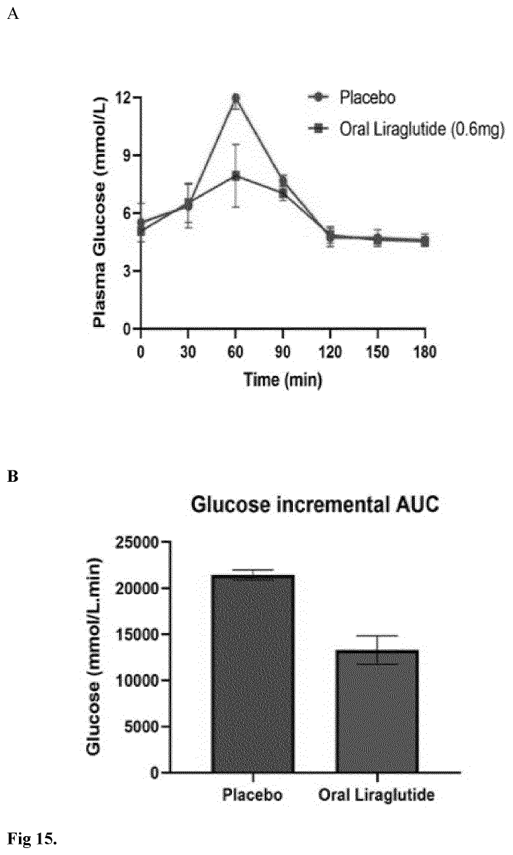

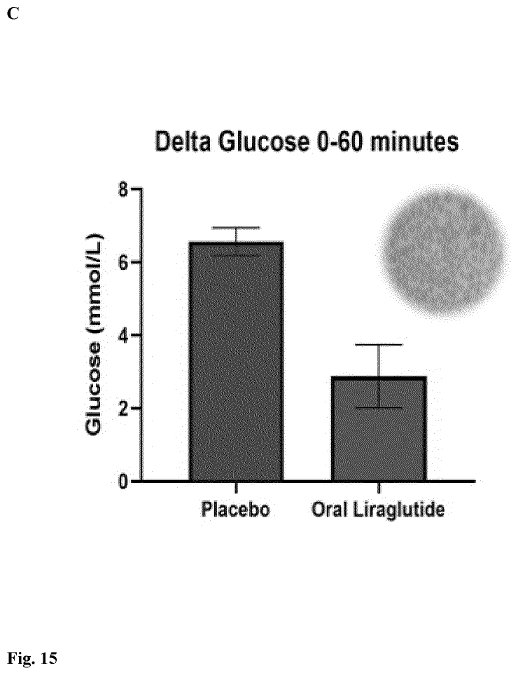

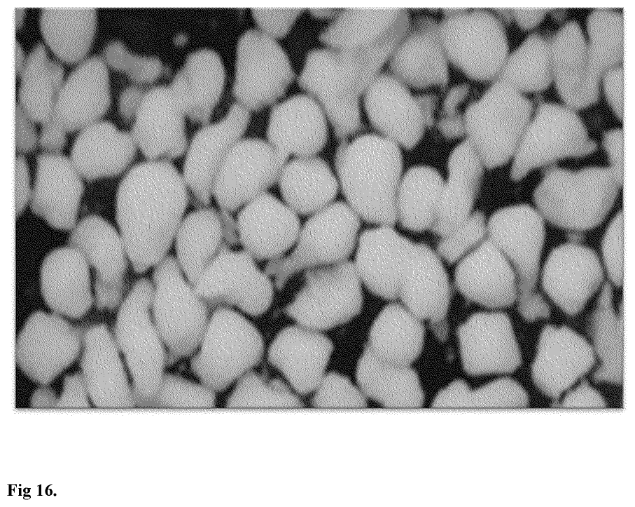

Further analysis was conducted at the end of testing (180 minutes incubation time), and spectrophotometric and chromatographic data verifies the release of mRNA nano-capsules from the encapsulated format into the basolateral side (FIG. 12). Detection of mRNA in the basolateral side verifies the absence of mRNA damage, which is protected by nano-particulates. This data indicates that mRNA nano-particles are released from micro-encapsulated formats in the luminal/apical side and subsequently permeates through the rat intestinal tissue to deliver stable nucleic acid nanoparticles to the basolateral side, within 180 minutes.

Free nucleic acids demonstrate low permeability across the gut epithelial tissue; however, micro- and nano-encapsulation provides a greater permeability and bioavailability for nucleic acids. It is important to note that the permeability after 180 minutes is marginally higher in ileal experiments relative to jejunal experiments. It is proposed that encapsulation technology provides greater sensitivity for nucleic acid permeation through the wall of the gut in the jejunal region.

Intestinal permeation enhancers are used to improve oral absorption of poorly permeable actives such as peptides or nucleic acids across the intestinal wall. Most pharmaceutical permeation enhancers such as sodium caprate (C10) are often associated but they provide with moderate damage to the cells that make up the intestinal epithelium. These cells have been shown to undergo rapid repair processes, however there is still the concern that long term, repeated exposure to permeation enhancers from pharmaceutical drug formulations may result in serious adverse effects to the intestinal epithelium.

Interestingly, protein matrices utilised for the creation of micro- and nano-encapsulation matrices are clean label and have previously demonstrated the presence of natural permeation enhancement effects; therefore it is postulated that these encapsulation formats could act as “adjuvants” to accentuate the permeation of nucleic acids into the blood and cell.

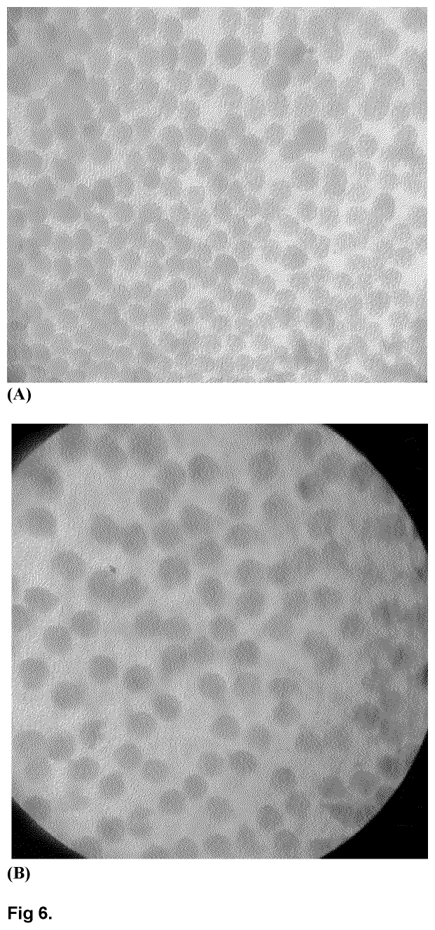

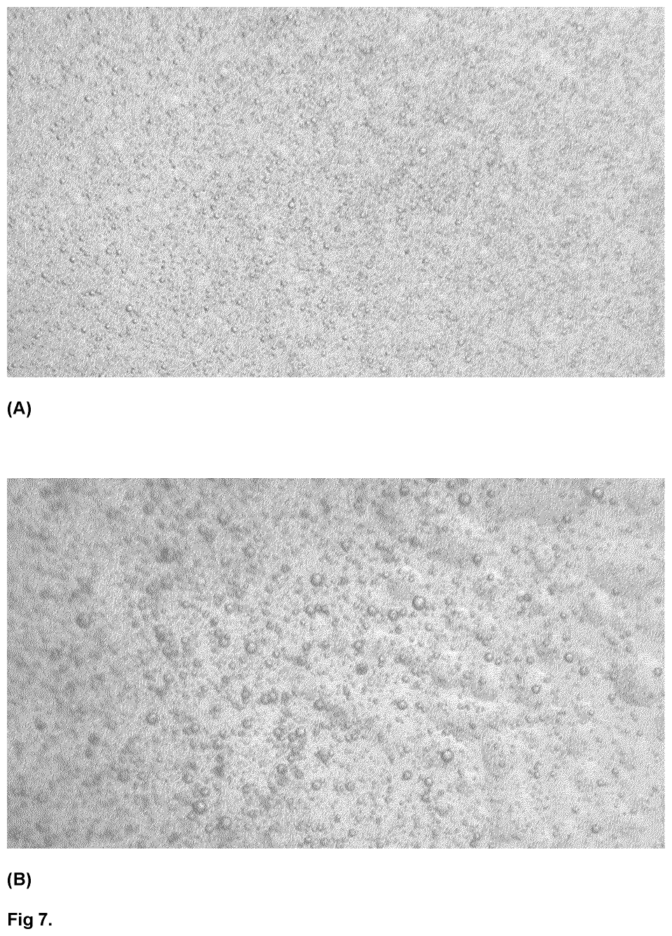

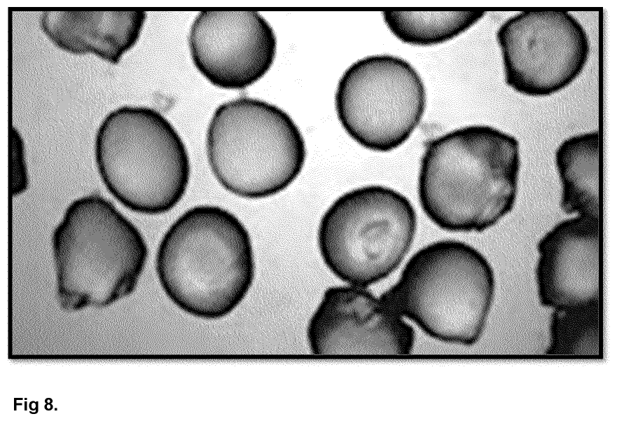

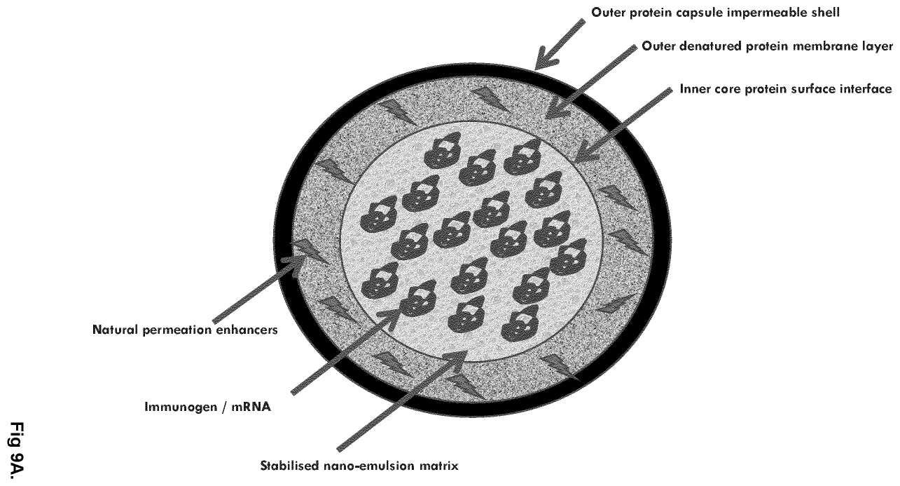

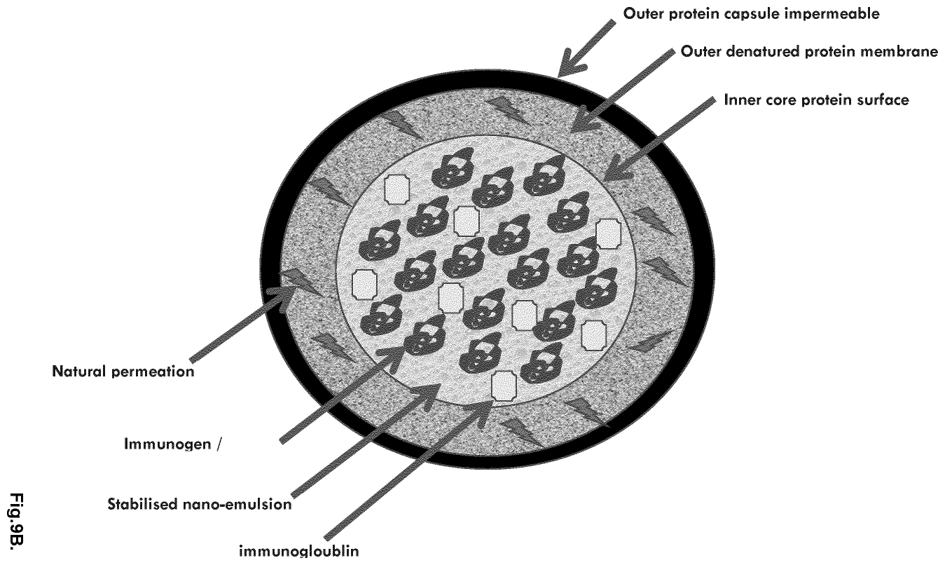

For bioavailability, it is important to note that a limit/saturation point for nucleic acid bioavailability is apparent as a function of time. FIG. 5 illustrates that there is a limit to the permeation and bioavailability that can be achieved with nucleic acids since the reduction in % TEER plateaus after 80 minutes. This result is related to the absorption pathway utilized in the body. Data demonstrates that micro-encapsulated nucleic acids permeate at an initial faster rate in the ileal region relative to jejunal region; however both achieved the same permeability kinetics after 100 min incubation (FIG. 10). This would indicate that there are i) no rate-limiting steps in the micro-encapsulated system to prevent nucleic acid bioavailability in the jejunum or ileum and ii) that nano-encapsulated nucleic acids are potentially absorbed and show equivalent bioavailability 100 min after digestion. Hence, micro- and nano-encapsulation does not appear to present any adverse rate limitation towards nucleic acid absorption. Moreover, apical-side exposure of the intestinal epithelium to micro-encapsulated nucleic acids did not cause damage or allergic reaction to the mucosa or sub-mucosa. Hence, this micro-encapsulated nucleic acid adjuvant can be classified as a safe and food grade material.

The ultra-high temperature denaturation process serves three purposes when denaturing the plant protein: (a) it kills any bacterial spores in the protein suspension rendering the protein of food-grade quality and ensuring they are safe for human consumption, (b) denaturation at this high of a temperature is necessary to provide the relevant functionality to polymerise plant proteins, and (c) it also generates a viscosity that permits for better flow rates during the encapsulation process resulting in higher efficiency in the encapsulation process and higher throughput during encapsulation.

In summary, micro-encapsulated nucleic acids can be absorbed and become highly bioavailable at a fast rate relative to free nucleic acids. Data illustrates an enhancement of bioavailability relative to free nucleic acids through the use of a combined system of micro- and nano-encapsulation technology. It is postulated that the digested micro-encapsulation protein matrix acts as a chaperon/permeation enhancer for the improved bioavailability and stabilisation of nucleic acids, delivered via nano-particles, to the basolateral region for potential rapid cell uptake.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. An oil in water nanoemulsion having an oil phase and an aqueous phase and comprising:
active pharmaceutical ingredient (API);
edible oil;
denatured plant protein;
surfactant; and
water,
in which the API is contained in the oil phase, and wherein the API comprises: at least one immunogen for use in vaccine therapy or a nucleic acid encoding the at least one immunogen; a hydrophobic drug; or a hormone, and the nucleic acid is an mRNA molecule.

2-3. (canceled)

4. The oil in water nanoemulsion according to claim 1, comprising 40 to 60% by weight water or aqueous buffer and 40 to 60% by weight solid ingredients, in which the solid ingredients comprise by weight:
55 to 95% API;
2 to 20% edible oil;
2 to 20% denatured plant protein;
1 to 5% surfactant.

5. (canceled)

6. The oil in water nanoemulsion according to claim 1, in which the solid ingredients comprise by weight 0 to 10% Immunoglobulin A.

7-8. (canceled)

9. The oil in water nanoemulsion according to claim 1, in which the plant protein is pea protein or mung bean protein.

10. A composition comprising:
5 to 20% by weight of the oil in water nanoemulsion according to claim 1; and
80 to 95% by weight of a denatured plant protein suspension.

11. The composition according to claim 10, further comprising a simple carbohydrate or a calcium salt chelating agent.

12. (canceled)

13. A method of forming a microparticle comprising an API encapsulated in a denatured plant protein matrix, the method comprising the steps of
preparing a protein suspension comprising denatured plant protein;
combining the protein suspension and the API to form a mixture;
treating the mixture to form a microparticle comprising API encapsulated in a denatured plant protein matrix, in which the treating step comprises polymerising the denatured plant protein matrix with a chelating salt or forming the microparticle by spray englobing on a fluidised bed dryer; and
drying the microparticle, wherein the API is provided in the form of an oil in water emulsion in which the API is contained in an oil phase of the oil in water emulsion.

14. (canceled)

15. The method according to claim 13, in which the protein suspension comprises a simple carbohydrate.

16. (canceled)

17. The method according to claim 13, in which the combining step comprises extruding the protein suspension and the oil in water nanoemulsion to form microdroplets, in which the polymerisation step comprises curing the extruded microdroplets in a curing bath comprising the chelating salt to form the microparticles, and including a step of drying the microparticles optionally by freeze-drying or vacuum drying.

18. The method according to claim 17, in which the protein suspension and the oil in water nanoemulsion are mixed prior to extrusion through a single nozzle extruder.

19. The method according to claim 17, in which the extruding employs a concentric nozzle extruder in which the protein suspension is extruded through the outer nozzle and the oil in water nanoemulsion is simultaneously extruded through the inner nozzle.

20. The method according to claim 13, in which the treating step comprises adding the chelating salt to the mixture to gel the mixture, drying the gelled mixture by freeze-drying or vacuum drying, and size-reducing the gelled mixture to provide the microparticles.

21. The method according to claim 20, in which the chelating salt comprises a calcium citrate buffer having a pH of 5 to 6.5 and a molarity of 0.05 to 0.15 M and/or the chelating salt is added to the mixture at a volumetric ratio of 1:100 to 1:300.

22-23. (canceled)

24. The method according to claim 13, in which the protein suspension comprises 0.5 to 5.0% simple carbohydrate and/or in which the combining step comprises combining the protein suspension and the oil in water nanoemulsion at a denatured plant protein to oil in water nanoemulsion weight ratio of 1:5 to 1:25.

25-26. (canceled)

27. The method according to claim 13, in which the method comprises forming the microparticles by spray englobing in which the combining and treating steps are performed on a fluidised bed dryer and, optionally, in which the method comprises adding a carrier material and an API to a bed of a fluidised bed dryer, fluidising the carrier material and API, spraying a first coating material onto the fluidised carrier material and API to produce a microparticles having an API and carrier contained within a shell of first coating material, and drying the microparticles and, optionally, in which the fluidised bed is fluidised with an airflow of 110 to 300 m³/hour.

28-31. (canceled)

32. The method according to claim 27, in which the method comprises spraying a chelating salt on the denatured plant protein coating.

33. The method according to claim 27, in which the spray englobing comprises the steps of:
(a) adding a carrier material and an API to a bed of a fluidised bed drying chamber;
(b) fluidising and heating the carrier material and API to form a first fluidised powder;
(c) spraying the denatured protein suspension onto the fluidised bed at elevated pressure to provide a second fluidised powder;
(d) drying the second fluidised powder on the fluidised bed to reduce the moisture content of the second fluidised powder
(e) spraying an englobing component on to the second fluidised powder to form a third fluidised powder, in which the englobing component is selected from a chelating salt, an edible oil, and a simple sugar;
(f) drying the third fluidised powder on the fluidised bed to further reduce the moisture content of the fluidised powder;
(g) spraying the denatured protein suspension onto the fluidised bed at elevated pressure to provide a fourth fluidised powder comprising microparticles; and,
optionally, further drying the fourth fluidised powder to a moisture content of less than 10%, 8%, or 5%.

34-35. (canceled)

36. The method according to claim 33, in which, the method comprises the following additional steps:
(h) bottom spraying denatured protein suspension onto the fourth fluidised mixture to form a fifth fluidised powder;
(i) drying the fifth fluidised powder on the fluidised bed to further reduce the moisture content of the fluidised powder;
(j) bottom spraying an englobing component on to the fifth fluidised powder to form a sixth fluidised powder, in which the englobing component is selected from a chelating salt and an edible oil;
(k) drying the sixth fluidised powder on the fluidised bed to further reduce the moisture content of the fluidised powder; and
(l) optionally, further drying the fourth fluidised powder to a moisture content of less than 10%, 8%, or 5%.

37-40. (canceled)

41. The method according to claim 33, in which the chelating salt is added as a 0.2-0.4 M solution.

42-43. (canceled)

44. The method according to claim 27, in which the spray englobing comprises the steps of:
fluidising a carrier mater, an API and a chelating salt on a fluidised bed of a fluidised bed drying chamber;
spraying an edible oil into the fluidised bed drying chamber;
spraying a suspension of denatured plant protein into the fluidised bed drying chamber, whereby the chelating salt reacts with the denatured plant protein to polymerise the protein and form agglomerated microparticles having a polymerised denatured protein coat and core comprising API and carrier material.

45-48. (canceled)

49. Microparticles obtained by the method of claim 13.

50. A microparticle comprising nanoparticulate drops of edible oil contained within a polymerised denatured plant protein structure, in which the drops of edible oil contain an active pharmaceutical ingredient (API).

51. A microparticle according to claim 50, in which the polymerised denatured plant protein structure is a polymerised denatured plant protein matrix, and the oil drops are distributed through the matrix.

52-54. (canceled)

55. The microparticle according to claim 50, further comprising a simple sugar, and, optionally, in which the microparticle is a granule.

56. (canceled)

57. A pharmaceutical composition in oral dose form comprising a microparticle of claim 50 in combination with a suitable pharmaceutical excipient and, optionally, in which the pharmaceutical composition is a vaccine composition and the API comprises an immunogen or a nucleic acid encoding an immunogen.

58. (canceled)

59. The pharmaceutical composition in oral dose form according to claim 57, in which the API is selected from an mRNA construct comprising mRNA encoding an immunogen, a hormone, and a hydrophobic drug.

60-62. (canceled)
